(19) United States
(12) Patent Application Publication
(10) Pub. No.: US 2004/0209828 A1
(43) Pub. Date: Oct. 21, 2004

(54) Solvent-free anthracycline derivatives

(76) Inventors: Alessandro Martini, Milan (IT); Massimo Zampieri, Cesano Maderno (IT); Paolo Fumagalli, Meda (IT); Ferdinando Giordano, Torre d' (IT); Ruggero Bettini, S Polo di Torrile (IT)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/484,512

(22) PCT Filed: Jul. 8, 2002
(86) PCT No.: PCT/EP02/07642

(30) Foreign Application Priority Data
Jul. 17, 2001 (GB) 0117403.6

(51) Int. Cl. C07H 15/24; A61K 31/704

(52) U.S. Cl. 514/34; 536/6.4

(57) ABSTRACT

There are provided a method for making highly purified crystals of anthracycline derivatives, and a new crystalline form of 4-demethoxy-3′-deamino-3′-aziridinyl-4′-methansulfonyl daunorubicin, useful in the treatment of tumors.

This invention relates to a method for making highly purified anthracycline derivatives, to a new crystalline form of an anthracycline, to a process for its preparation and to pharmaceutical composition containing it. The invention features the use of supercritical fluid in the preparation of highly purified crystalline forms of anthracycline derivatives without appreciable content of residual solvents. In addition, the present invention provides a new crystal phase of an anthracycline.

High quality, pure crystals are of great value for a variety of industrial and research applications in the field of anthracyclines, a well known class of antitumor drugs. For example, a frequent problem in pharmaceutical research is the difficulty of obtaining large single crystals for X-ray structural analyses to determine molecular conformations. Crystallization is also frequently used as a method of purification in the biotechnology and pharmaceutical industries. More highly ordered crystals will typically result in a more highly purified product. Larger crystals can also impart advantages in packaging and handling operations. Conventional crystal purification methods are generally restricted to molecule that are thermally stable at high temperatures, and this common drying techniques can be ineffective for thermally unstable and crystalline solvated compounds.

Over the past decade, compressed gases, liquefied gases, and materials intermediate to gases and liquids known as supercritical fluids have been used as anti-solvents. A pure compound becomes critical at its critical temperature (T.sub.c) and critical pressure (P.sub.c). A compound becomes a supercritical fluid above its critical temperature and at its critical pressure, or above its critical pressure and at its critical temperature, or where conditions exceed both the critical temperature and pressure. These parameters are intrinsic thermodynamic properties of all sufficiently stable pure component compounds. Carbon dioxide, for example, becomes a supercritical fluid at conditions equal to or exceeding its critical temperature of 31.1° C. and its critical pressure of 72.8 atm (1,070 psig, 7.38 megapascals). In the supercritical region, normally gaseous compounds exhibit greatly enhanced salvation power. At a pressure of 3,000 psig (204 atm, 20.7 megapascals) and a temperature of 40°C, carbon dioxide has a density around 0.8 g/ml and behaves very much like a nonpolar organic solvent, with a zero dipole moment.

Our International patent EP01/02783 application filed on Mar. 12, 2001 describes and claims a crystal form of 4-demethoxy-3′-deamino-3′-aziridinyl-4′-methansulfonyl daunorubicin, which is referred to as “alkycycline” hereinafter and is a valuable antitumor drug, obtained from different solvents. For example, one of the crystallisation methods there described was based on a cooling (0-4° C.) from a concentrated solution of raw alkycycline in ethyl acetate subsequently filtered and dried at 30° C. under vacuum for about two days. This process allowed manufacturing batches showing increased solid state stability, and the content of residual ethyl acetate was eliminated by drying at less than 50° C. under reduced pressure to avoid chemical degradation and solid state modification of the compound. Nevertheless, these conditions are not suitable to dry development scale batches under GMP (Good Manufacture Production) conditions. On the other hand, it was assessed that current drying techniques were not efficacious for a complete solvent removal and prolonged time of storage at 40° C. under vacuum can determine partial degradation. Ethyl Acetate is an ICH class 3 solvent, thus its presence could be justified “ . . . provided that it is realistic in relation to manufacturing capability and good manufacturing practice” (ICH-1997). However, the effects of the presence of such a level of solvent can hinder the possibility to develop formulations where the solvent can interact with other excipients or with, the primary package (e.g. hard gelatin capsules) altering the formulation performances with ageing.

Therefore, the present invention provides a new process for preparing highly dried crystal of anthracycline derivatives, based on the employing of supercritical fluid at different condition of pressure and temperature in order to fully extract the residual solvents and the crystal compositions so formed. One embodiment of the present method for forming crystals comprises the steps of providing a compound of 96-100% purity. Preferably the supercritical fluid is selected from the group of gases capable of forming supercritical fluid consisting of nitrous oxide, propane and other light alkanes, ethylene and other light alkenes, fluorocarbons, chlorofluorocarbons, and carbon dioxide. SC—CO₂ is the preferred supercritical fluid.

The supercritical fluid extraction process can be carried out by means of an apparatus composed by a fluid reservoir pumped by means of a membrane pump into a thermostated column where extraction is performed. The fluid evolving from the column is expanded by means of a micrometric valve and then sank in a vessel containing a solvent like EtOH to recover possible spillage of dissolved drug from the column filling. The process can be performed at different temperatures, pressures and flow rates in order to set up proper conditions. For example, the SC—CO₂ process can be carried out at a temperature of from 25° to 55° C., pressure from 10 to 60 megapascals, for a period of about 3 hrs with a flow rate of from 50 ml/min to 4 l/min, measured beyond the micrometric expansion valve. More preferred purification conditions are temperature of from 40° to 45°, 30 megapascals, flow rate of 100 to 800 ml/min, still more preferred 400 to 800 ml/min. Aliquots of the anthracycline derivative are sampled into the column. The powders can be previously mixed with an inert filling (small glass spheres) to prevent packing and allowing the highest surface available for the SC treatment.

A further embodiment of the present invention comprises a new crystalline alkycycline, its preparation and its usage as an anticancer agent.

X-RAY Powder Diffraction

Powder X-ray diffraction was performed using a Siemens D-500 apparatus, irradiating powder samples with a CuKα graphite-monochromatic (40 kV 40 mA) source between 5° and 35° (2θ) at room temperature. The scan was made of 0.050 steps and the count time was 7 seconds per step.

The x-ray powder diffraction pattern for crystalline alkycycline shows a crystalline structure with distinctive peaks as shown in the following table I (obtained in the experimental conditions applied to the column 3—see example 1)

TABLE I

| Angle (°2θ) | Relative Intensity |
|---|---|
| 6.25 | 100.0 |
| 8.50 | 20.1 |
| 10.40 | 43.8 |
| 12.20 | 24.1 |
| 14.45 | 41.5 |
| 18.25 | 44.1 |
| 18.45 | 57.3 |

Analytical Methods Description Thermogravimetric Analyses (TGA)

TGA analyses were carried out with a Perkin-Elmer TGA-7. The temperature range of analysis was between 35° and about 210° C. Sodium tartrate was used to assess the proper performance of the apparatus with regard to the mass loss determination while ferromagnetic standards (Alumel alloy and Nickel) were used for temperature calibration. The samples were analyzed under nitrogen flow (to enhance desolvation and eliminate oxidative effects) at a heating rate of 2° C./min. Thermal range between 50° and 155° C has been considered and weight loss considered parameter of interest. Sampling was carried out using aluminum pans (Mettler ME-27331) of known weight, hermetically sealed. The sample pans were perforated with pin before starting the analysis.

High Performance Liquid Cromatography (HPLC)

The following experimental conditions were employed.

Materials

Ammonium Formate, analytical grade

Formic acid, analytical grade

Tetrahydrofuran (THF), HPLC grade

Distilled water, HPLC grade

Equipment

Waters model 600 liquid chromatograph or equivalent equipped with: Degassing system, Autosampler Waters model 717 Plus or equivalent, UV diode array detector Waters model PDA 966 or equivalent, Personal computer with Waters Millennium³² software, Column Symmetry Shield C8 (Waters), 3.5 μm, 100×4.6 mm

Sample Solution

Weigh accurately about 5 mg of sample under analysis, transfer into a 50 ml volumetric flask, dissolve and bring to volume with a mixture H₂O:THF in the ratio 50:50 immediately before analysis.

Chromatographic Conditions 20 μl of the sample solution is injected into the liquid chromatograph under the following experimental conditions:

| | |
|---|---|
| Column temperature | 40° C. ± 1° C. |
| Mobile phase flow rate | 1.0 ml/min |
| Analytical wavelength | 254 ± 1 nm |
| Mobile phase A | Formate buffer (50 mM HCOONH₄) made |
| | up to pH 4.5 with 10% formic acid |
| Mobile phase B | THF |
| Gradient program | Isocratic elution at 35% B for 15 |
| | minutes, followed by linear gradient |
| | to 55% B in 10 minutes, and still |
| | isocratic elution at 55% B to the |
| | end time (35 minutes); reequilibration |
| | time: 10 minutes. |

The crystalline alkycycline of the present invention can be produced by removing the residual solvent through SC—CO₂ process carried out at a pressure of more than 38 megapascals, starting from a batch of alkycycline, prepared by classic crystallisation as described in International patent application EP01/02783.

A further aspect of the present invention is to provide a method of treating a mammal including humans, suffering from a neo-plastic disease state comprising administering to said mammal a pharmaceutical composition obtained starting from the crystalline alkycycline as defined above in amounts effective to produce a anti-cancer effect.

By the term “administered” or “administering” as used herein is meant parenteral and /or oral administration. It will be appreciated that the actual preferred method and order of administration will vary according to, inter alia, the particular formulation of the alkycycline being utilized, the particular tumors being treated, and the particular host being treated.

In the method of the subject invention, for the administration of the alkycycline formulation, the course of therapy generally employed is from about 0.1 to about 200 mg/m² of body surface area. More preferably, the course therapy employed is from about 1 to about 50 mg/m² of body surface area.

The anti-neoplastic therapy of the present invention is in particular suitable for treating breast, ovary, lung, colon, kidney and brain tumors in mammals, including humans. The invention may be more fully understood with reference to the following examples.

### EXAMPLE 1

In order to perform the SC—CO₂ extraction process, aliquots of about 500 mg of alkycycline were sampled into HPLC columns.

The following stainless steel blank columns were employed to prepare four samples suitable for SC—CO₂ extraction.

| Size (internal - cm) | Total volume (ml) | Column No. |
|---|---|---|
| 24.5(l) × 0.36(d) | 2.49 | 1 |
| 25(l) × 0.46(d) | 4.15 | 2-3-4 |

Small glass spheres (0.5÷1 mm)—previously mixed with the samples—were used as an inert filling of the columns. The process was carried out in three hours, values related to each column were reported in the following table with notes about macroscopic properties of the recovered powders.

| | Recovery | | |
|---|---|---|---|
| | after | | |
| | process | Supercritical | |
| Column # | (mg) | parameters | Flow rate |
| 1 | 385 | 40° C. - 30 megapascals | 100 ml/min |
| 2 | 311 | 45° C. - 40 megapascals | 400-800 |
| | | | ml/min |
| 3 | 330 | 40° C. - 40 megapascals | 500 ml/min |
| 4 | 354 | 45° C. - 30 megapascals | 500 ml/min |

After the receiving of each column, the filling was taken out and separated from the glass spheres by sieving in a glove box. Aliquots were prepared for the different analyses.

The analytical values (HPLC assay, TGA weight loss and residual ethyl acetate content) are reported in the following table:

| | | AcOEt | HPLC Assay |
|---|---|---|---|
| | | Initial | Initial |
| Column | TGA | values in | values in |
| No. | Weight Loss % | brackets | brackets |
| 1 | 50-155° C.: 3.0 | 0.95% | 90.98% |
| | | (2.4) | (93.07) |
| 2 | 50-155° C.: 0.2 | 60 ppm | 92.61% |
| | | (2.4) | (93.07) |
| 3 | 50-155° C.: ≦0.1 | 30 ppm | 94.05% |
| | | (1.46) | (94.60) |
| 4 | 50-155° C.: ≦0.1 | Not | 95.28% |
| | | Detectable | (94.60) |
| | | (1.46) | |

X-Rays Powder Diffraction obtained in the experimental conditions applied to the column 4 are shown in the following table II:

TABLE II

| Angle (°2θ) | Relative Intensity |
|---|---|
| 5.8 | 44.2 |
| 7.15 | 100.0 |
| 12.95 | 25.5 |
| 14.20 | 39.8 |
| 19.60 | 24.8 |
| 23.05 | 22.0 |
| 26.90 | 14.0 |

1. A process for obtaining highly purified crystals of anthracycline derivatives with the use of supercritical fluid.

2. A process according to claim 1 in which the anthracycline derivative is 4-demethoxy-3′-deamino-3′-aziridinyl-4′-methansulfonyl daunorubicin.

3. A process according to claim 1 in which the supercritical fluid is carbon dioxide.

4. A process according to claim 1 wherein the process is carried out at a temperature of from 25° to 55° C., pressure of from 10 to 60 megapascals, for a period of about 3 hrs with a flow rate of from 50 ml/min to 4 l/ml.

5. A process according claim 1 wherein there is obtained a solvent free crystalline form of 4-demethoxy-3′-deamino-3′-aziridinyl-4′-methansulfonyl daunorubicin having distinctive peaks in powder X-ray diffraction as shown in the following table II:
TABLE II

| Angle (°20) | Relative Intensity |
|---|---|
| 5.8 | 44.2 |
| 7.15 | 100.0 |
| 12.95 | 25.5 |
| 12.20 | 39.8 |
| 19.60 | 24.8 |
| 23.05 | 22.0 |
| 26.90 | 14.0 |

6. A solvent free crystalline form of 4-demethoxy-3′-deamino-3′-aziridinyl-4′-methansulfonyl daunorubicin having distinctive peaks in powder X-ray diffraction as shown in the following table I:
TABLE I

| Angle (°20) | Relative Intensity |
|---|---|
| 6.25 | 100.0 |
| 8.50 | 20.1 |
| 10.40 | 43.8 |
| 12.20 | 24.1 |
| 14.45 | 41.5 |
| 18.25 | 44.1 |
| 18.45 | 57.3 |

7. A process for preparing a crystalline form of 4-demethoxy-3′-deamino-3′-aziridinyl-4′-methansulfonyl daunorubicin as defined in claim 6 characterized in that supercritical fluid extraction is carried out at a temperature of from 40° to 45° C., pressure from 38 to 60 megapascals, for a period of about 3 hrs with a flow rate of from 400 to 800 ml/min.

8. A method of treatment of cancer in a human or animal comprising administering a crystalline form of 4-demethoxy-3′-deamino-3′-aziridinyl-4′-methansulfonyl daunorubicin as defined in claim 6 to a human or animal in need thereof.

9. Use of a crystalline form of 4-demethoxy-3′-deamino-3′-aziridinyl-4′-methansulfonyl daunorubicin as in defined in claim 6 in the preparation of a medicament for use in the treatment of cancer in a patient in need of such treatment.

10. A pharmaceutical composition containing a crystalline form of 4-demethoxy-3′-deamino-3′-aziridinyl-4′-methansulfonyl daunorubicin as defined in claim 6 and pharmaceutically acceptable diluent of carrier.

11. A method of treating cancer comprising administrating to a person in need thereof, a pharmaceutical composition according to claim 10.
